(19) United States
(12) Patent Application Publication
(10) Pub. No.: US 2013/0157360 A1
(43) Pub. Date: Jun. 20, 2013

(54) BIOMIMETIC TISSUE SCAFFOLD AND METHODS OF MAKING AND USING SAME

(71) Applicants: John C. March, Ithaca, NY (US); Jiajie Yu, Ithaca, NY (US); Jong Hwan Sung, Ithaca, NY (US)

(72) Inventors: John C. March, Ithaca, NY (US); Jiajie Yu, Ithaca, NY (US); Jong Hwan Sung, Ithaca, NY (US)

(73) Assignee: CORNELL UNIVERSITY, ITHACA, NY (US)

(21) Appl. No.: 13/806,225

(22) PCT Filed: Jun. 24, 2011
(86) PCT No.: PCT/US2011/041746

§ 371 Date: Mar. 6, 2013

Related U.S. Application Data
(60) Provisional application No. 61/358,613, filed on Jun. 25, 2010.

(51) Int. Cl. A61L 27/14

(52) U.S. Cl. CPC A61L 27/14
USPC 435/366; 435/396; 264/334; 264/400

(57) ABSTRACT

Three-dimensional biomimetic tissue scaffolds, as well as methods of manufacture of these scaffolds. The method is fully customizable to create a biomimetic tissue scaffold with shapes, densities, and geometries similar or identical to the tissue it imitates. For example, physiologically realistic collagen/PEG villi created using the method are designed to have a high-aspect ratio and curvature similar to villi found in the human small intestine. Accordingly, the biomimetic tissue scaffolds serve as an improved in vitro model for a wide variety of physiological research, as well as pharmacological testing and drug, compound, and/or metabolite uptake by cells growing on the scaffold, among many other uses.

### CROSS-REFERENCE TO RELATED APPLICATIONS

The present PCT application claims the priority of U.S. Provisional Application No. 61/358,613 entitled “Artificial Villi and Methods of Making and Using Same” filed on Jun. 25, 2010, the entire contents of which are incorporated herein by reference.

### BACKGROUND OF THE INVENTION

1. Field of the Invention

The present invention relates to a biomimetic tissue scaffold, and, more particularly, to a biomimetic tissue scaffold comprising a hydrogel or other polymeric material, as well as methods of manufacture and use of the biomimetic tissue scaffold.

2. Description of the Related Art

The ability to achieve authentic tissue function in vitro is important not only from purely scientific point of view, but also in application-oriented areas such as tissue engineering and pharmaceutical development. It is well-known that traditional two-dimensional (“2D”) cell cultures can be significantly different from their in vivo counterparts, and recently it has been demonstrated that cells exhibit more authentic functions if a physiologically realistic environment is provided. In particular, a three-dimensional (“3D”) cell culture allows for more physiologically relevant cell-to-cell and cell-to-matrix interactions, as well as proper chemical and mechanical signaling.

As the use of 3D cell culture grows, various hydrogels have been developed as scaffolds for 3D cell culture. Hydrogels are hydrophilic polymers, with their major fraction being water, and thus provide a cell-friendly environment as well as mechanical support for cell growth and differentiation. Typically, cells are encapsulated within or cultured on the surface of these hydrogels. For these cell-laden hydrogels to correctly reproduce the biological functions of in vivo tissues, it is important to accurately mimic the three-dimensional geometry of the native tissue in micro/nanometer resolution, such that cells can be induced to behave in a more authentic manner.

A large number of synthetic and naturally-derived hydrogels exist, with a wide range of mechanical and chemical properties. Some known natural polymers and synthetic monomers used in hydrogel fabrication include chitosan, alginate, fibrin, collagen, gelatin, hyaluronic acid, dextran, hydroxyethyl methacrylate, N-(2-hydroxypropyl) methacrylate, N-vinyl-2-pyrrolidone, N-isopropyl acrylamide, vinyl acetate, acrylic acid, methacrylic acid, polyethylene glycol acrylate/methacrylate, and polyethylene glycol diacrylate/dimethylacrylate, just to name a few.

Several methods have been developed to construct microscale tissue geometries with hydrogels, such as replica molding, photo-polymerization, and direct printing. However, these methods are typically limited to the fabrication of low to medium aspect ratio structures, often with perpendicular shapes. The “aspect ratio” of a structure or three-dimensional shape is the ratio of its longer dimension (or axis) to its shorter dimension (or axis), and a “high-aspect ratio” indicates that the longer dimension (or axis) is greater than the shorter dimension (or axis). It is technically challenging to fabricate more complex structures, such as a structure with a high aspect ratio or curvature. For example, intestinal villi typically have cone-shaped, high aspect ratio morphology. Conventional replica molding is not suitable for fabrication of such shapes, since detaching the soft hydrogel scaffold from a mold results in destruction of the structure. While the photo-polymerization method has a resolution of several micrometers, it cannot create curved 3D shapes, and is limited to photo-polymerizable hydrogels only. Direct printing methods are suitable for free-form fabrication of arbitrary shapes, but are typically suitable for low-resolution applications (hundreds of micrometers), cannot make curved shapes, and require expensive equipment.

For example, there is a continued need for a suitable three-dimensional model to study the human gastrointestinal (“GI”) tract, including the small intestine. The small intestine performs most of the chemical digestion and absorption in the body by breaking down proteins, lipids, and carbohydrates and then absorbing these nutrients through millions of projections from the intestinal wall called villi. The villi contain blood vessels that carry these nutrients to the rest of the body. Growing along these villi are four types of epithelial cells: enterocytes (absorption), enteroendocrine (hormone secretion), goblet (mucus production), and Paneth cells (phagocytosis). Paneth cells, for example, are targets for drug delivery because of their phagocytic characteristics and their role in regulating the microbial population of the small intestine. Enterocytes, on the other hand, participate in the process of oral absorption, by which unchanged drug molecules proceed from site of administration, such as the mouth and the gut lumen, to the site of measurement within the body. The extent of oral absorption depends on the extent of first-pass elimination in the gut wall and liver.

Current artificial or synthetic GI models are primarily 2D, with little resemblance of the physical arrangement, definition and contents of the intestine. One model system is the 2D cell insert configuration, in which cells are grown on culturing inserts that are placed in well plates such that the 2D culture layer is exposed to different media on its basolateral and apical sides. This system can be seeded with a second monolayer on the basolateral side which can serve as a tissue and is closer to that which is found in the gut than other 2D systems, but lacks the 3D architecture of the villi and does not allow for basophils and epithelia to be linked by a tissue layer as is seen in the actual upper intestine.

Despite these many recent advances in 3D cell culture scaffold, there is a continued need for affordable 3D cell culture scaffolds with complex geometries similar to those found in nature, including structures with curvature and/or a high-aspect ratio.

### SUMMARY OF THE INVENTION

An embodiment of the invention is directed to a biomimetic tissue scaffold.

Another embodiment of the invention is directed to a biomimetic tissue scaffold comprising a hydrogel or other polymeric material.

Another embodiment of the invention is directed to a 3D tissue scaffold comprising complex geometries similar to those found in nature, including structures with curvature and/or high-aspect ratio morphology.

Yet another embodiment of the invention is directed to an artificial intestinal model including a villi scaffold in which the villi comprise curvature and a high-aspect ratio similar to human intestinal villi.

A further embodiment of the invention is directed to an artificial intestinal model capable of bearing a cell culture.

Another embodiment of the invention is directed to an efficient and affordable method of producing a biomimetic tissue scaffold.

Other embodiments of the present invention will in part be obvious, and in part appear hereinafter.

According to various aspects of the invention is provided a method for making a three-dimensional biomimetic scaffold capable of supporting growth of a cell, the method comprising the steps of: (i) forming a first three-dimensional shape in a first mold; (ii) filling at least a portion of the three-dimensional shape in the first mold with a first polymerizable compound; (iii) causing the first polymerizable compound to polymerize to form a three-dimensional scaffold, where the three-dimensional scaffold is complementary to the three-dimensional shape; and (iv) removing the three-dimensional scaffold from the first mold. In one embodiment, the three-dimensional shape is formed using laser ablation. The three-dimensional shape can be any shape, including but not limited to a plurality of three-dimensional indentations, where a majority of the indentations has a maximum height that is greater than a maximum width.

According to a second aspect of the invention is provided a method for making a three-dimensional biomimetic scaffold capable of supporting growth of a cell, the method comprising the steps of: (i) forming a first three-dimensional shape in a first mold; (ii) filling at least a portion of the three-dimensional shape in the first mold with a first polymerizable compound; (iii) causing the first polymerizable compound to polymerize to form a three-dimensional scaffold, where the three-dimensional scaffold is complementary to the three-dimensional shape; (iv) removing the three-dimensional scaffold from the first mold; and (v) seeding the first polymerizable compound with a cell at some point prior to the step of causing the first polymerizable compound to polymerize to form a three-dimensional scaffold.

According to a third aspect of the invention is provided a method for making a three-dimensional biomimetic scaffold capable of supporting growth of a cell, the method comprising the steps of: (i) filling at least a portion of a three-dimensional shape formed in a first mold with a first polymerizable compound; (ii) causing the first polymerizable compound to polymerize to form a second mold, wherein at least a portion of the second mold comprises a first structure which is complementary to the three-dimensional shape; (iii) removing the second mold from the first mold; (iv) using the second mold to form a third mold from a second polymerizable compound; (v) removing the third mold from the second mold; and (vi) using the third mold to form a three-dimensional scaffold from a third polymerizable compound, wherein the three-dimensional scaffold is complementary to the three-dimensional shape. In one embodiment, the method further comprises the step of removing the third mold away from the three-dimensional scaffold. In a further embodiment, the method further comprises the step of forming the first three-dimensional shape in the first mold. In an embodiment, the first mold comprises a plastic such as poly(methyl methacrylate), the first polymerizable compound comprises a silicone such as polydimethylsiloxane, the second polymerizable compound comprises gelatin hydrogel, alginate, gelatin, chitosan, collagen, poly-N-isopropylacrylamide, a polysaccharide-based polymer, poly(ethylene glycol), poly(ethylene glycol)diacrylate, or a combination thereof, and the third polymerizable compound comprises a hydrogel compound such as collagen/PEG-DA, or a non-hydrogel compound such as, for example, polycarbonate.

According to a fourth aspect of the invention is provided the first mold as described above, wherein the three-dimensional shape is formed using laser ablation. The three-dimensional shape can be, for example, a plurality of indentations. In one embodiment, each of the indentations has a maximum height and a maximum width, and for most of the indentations the maximum height of the indentation is greater than the maximum width of the indentation. For example, the height of the villi can range from 50 μm to 5 mm, and the width can range from 5 μm to 5 mm. The indentations can also have a conical shape, similar to intestinal villi, or a wide variety of other shapes (including cylindrical, dumbbell, or mushroom, among others).

According to a fifth aspect of the invention is provided a method for making a three-dimensional biomimetic scaffold capable of supporting growth of a cell, the method comprising the steps of: (i) filling at least a portion of a three-dimensional shape formed in a first mold with a first polymerizable compound; (ii) causing the first polymerizable compound to polymerize to form a second mold, wherein at least a portion of the second mold comprises a first structure which is complementary to the three-dimensional shape; (iii) removing the second mold from the first mold; (iv) using the second mold to form a third mold from a second polymerizable compound; (v) removing the third mold from the second mold; (vi) using the third mold to form a three-dimensional scaffold from a third polymerizable compound, wherein the three-dimensional scaffold is complementary to the three-dimensional shape; and (vii) seeding the third polymerizable compound with a cell at some point prior to the step of using the third mold to form the three-dimensional hydrogel scaffold.

According to a sixth aspect of the invention is provided a method for making a three-dimensional biomimetic scaffold capable of supporting growth of a cell, the method comprising the steps of: (i) filling at least a portion of a three-dimensional shape formed in a first mold with a first polymerizable compound; (ii) causing the first polymerizable compound to polymerize to form a second mold, wherein at least a portion of the second mold comprises a first structure which is complementary to the three-dimensional shape; (iii) removing the second mold from the first mold; (iv) using the second mold to form a third mold from a second polymerizable compound; (v) removing the third mold from the second mold; (vi) using the third mold to form a three-dimensional scaffold from a third polymerizable compound, wherein the three-dimensional scaffold is complementary to the three-dimensional shape; (vii) seeding the three-dimensional scaffold with one or more cells; and (viii) incubating the cell(s).

According to a seventh aspect of the invention is provided a method for making a three-dimensional biomimetic scaffold capable of supporting growth of a cell, the method comprising the steps of: (i) filling at least a portion of a three-dimensional shape formed in a first mold with a first polymerizable compound; (ii) causing the first polymerizable compound to polymerize to form a second mold, wherein at least a portion of the second mold comprises a first structure which is complementary to the three-dimensional shape; (iii) removing the second mold from the first mold; (iv) using the second mold to form a third mold from a second polymerizable compound; (v) removing the third mold from the second mold; (vi) using the third mold to form a three-dimensional scaffold from a third polymerizable compound, wherein the three-dimensional scaffold is complementary to the three-dimensional shape; and (vii) using the three-dimensional scaffold for pharmacological testing, to examine a biological process, for toxicology studies, or for stem cell studies.

According to a eighth aspect of the invention is provided a system for making a three-dimensional biomimetic scaffold capable of supporting growth of a cell, the system comprising: (i) a first mold comprising a three-dimensional shape; (ii) a second mold formed from the first mold using a first polymerizable compound; and (iii) a third mold formed from the second mold using a second polymerizable compound, where the third mold is used to make a three-dimensional scaffold complementary to the three-dimensional shape. In one embodiment, the first three-dimensional shape comprises a plurality of indentations. Each of the indentations has a maximum height and a maximum width, and for most of the indentations, the maximum height is greater than the maximum width. In another embodiment, the second polymerizable compound is selected from the group consisting of a hydrogel, alginate, gelatin, chitosan, collagen, poly-N-isopropylacrylamide, a polysaccharide-based polymer, poly(ethylene glycol), poly(ethylene glycol)diacrylate, or a combination thereof, and the third polymerizable compound comprises a hydrogel compound such as, for example, collagen/PEG-DA, or a non-hydrogel compound such as, for example, polycarbonate.

According to a ninth aspect of the invention is provided a system for making a three-dimensional biomimetic scaffold capable of supporting growth of a cell, the system comprising: (i) a first mold comprising a three-dimensional shape; (ii) a second mold formed from the first mold using a first polymerizable compound; (iii) a third mold formed from the second mold using a second polymerizable compound, where the third mold is used to make a three-dimensional scaffold complementary to the three-dimensional shape; and (iv) a cell seeded on or in the three-dimensional scaffold.

According to an tenth aspect of the invention is provided a three-dimensional biomimetic scaffold formed by the following steps: (i) filling at least a portion of a three-dimensional shape formed in a first mold with a first polymerizable compound; (ii) causing the first polymerizable compound to polymerize to form a second mold, wherein at least a portion of the second mold comprises a first structure which is complementary to the three-dimensional shape; (iii) removing the second mold from the first mold; (iv) using the second mold to form a third mold from a second polymerizable compound; (v) removing the third mold from the second mold; and (vi) using the third mold to form a three-dimensional scaffold from a third polymerizable compound, wherein the three-dimensional scaffold is complementary to the three-dimensional shape. In one embodiment, the final scaffold comprises a three-dimensional villi structure made from a polymerized hydrogel compound, although non-hydrogel compounds may also be utilized.

According to an eleventh aspect of the invention is provided a three-dimensional biomimetic scaffold comprising a cell seeded or in the scaffold, where the scaffold is formed via the following steps: (i) filling at least a portion of a three-dimensional shape formed in a first mold with a first polymerizable compound; (ii) causing the first polymerizable compound to polymerize to form a second mold, wherein at least a portion of the second mold comprises a first structure which is complementary to the three-dimensional shape; (iii) removing the second mold from the first mold; (iv) using the second mold to form a third mold from a second polymerizable compound; (v) removing the third mold from the second mold; and (vi) using the third mold to form a three-dimensional scaffold from a third polymerizable compound, wherein the three-dimensional scaffold is complementary to the three-dimensional shape

According to a twelfth aspect of the invention is provided a method for making an intestinal reactor, the method comprising the steps of: (i) forming a biomimetic scaffold comprising a plurality of villi; (ii) seeding at least one of said villi with a cell; and (iii) forming a hollow tube from the seeded biomimetic scaffold, where the hollow tube has an interior surface and an exterior surface. The villi can be located on either the interior or the exterior surface of the reactor, depending on the desired use or application. In one embodiment, the method further comprises the steps of: adding a microorganism to the intestinal reactor; and/or adding nutrients to the intestinal reactor. In yet another embodiment, the method further comprises the steps of: using the intestinal reactor for pharmacological testing; and/or using the intestinal reactor to examine an intestinal process.

According to an thirteenth aspect of the invention is provided an intestinal reactor formed by a method comprising the steps of: (i) forming a biomimetic scaffold comprising a plurality of villi; (ii) seeding at least one of said villi with a cell; and (iii) forming a hollow tube from the seeded biomimetic scaffold, where the hollow tube has an interior surface and an exterior surface. The villi can be located on either the interior or the exterior surface of the reactor, depending on the desired use or application.

### BRIEF DESCRIPTION OF THE DRAWINGS

The present invention will be more fully understood and appreciated by reading the following Detailed Description in conjunction with the accompanying drawings, in which:

FIG. 1 is flowchart showing an exemplary method for producing a biomimetic tissue scaffold according to one embodiment of the present invention;

FIG. 2 is a representative schematic showing a method for producing a hydrogel tissue scaffold seeded with cells according to one embodiment of the present invention;

FIG. 3 is a flowchart showing an exemplary method for producing a biomimetic tissue scaffold according to one embodiment of the present invention;

FIG. 4A is a confocal microscope image of a collagen scaffold after three-dimensional rendering;

FIG. 4B is a confocal microscope image of a PEG scaffold after three-dimensional rendering;

FIG. 5A is a confocal microscope image of Caco-2 cells seeded and incubated on a scaffold, after staining for actin and nucleic acid;

FIG. 5B is a confocal microscope image (X-Y slice) of Caco-2 cells on a scaffold, stained for actin and nucleic acid;

FIG. 6A is a confocal microscope image of a collagen scaffold after three-dimensional rendering, in which the scaffold is covered with Caco-2 cells;

FIG. 6B is a confocal microscope image of a collagen scaffold after three-dimensional rendering, in which the scaffold has not been seeded with cells;

FIG. 7A is a confocal microscope image of Caco-2 cells on a PDMS scaffold;

FIG. 7B is a confocal microscope image of Caco-2 cells on a PDMS scaffold;

FIG. 8 is a graph of the measured depth of the indentations formed in a PMMA mold using pulsed laser, versus the laser pulse number;

FIG. 9A is an image of a poly(methyl methacrylate) (“PMMA”) mold after exposure to laser pulses to create indentations at a density of 25/mm²;

FIG. 9B is an image of the reverse side of the PMMA mold of FIG. 7A with the camera focused on the bottom of the indentations;

FIG. 10 is a scanning electron microscope image of a polydimethylsiloxane (“PDMS”) structure made from a PMMA mold similar to the PMMA mold depicted in FIGS. 7A and 7B;

FIG. 11 is a schematic representation of the formation of a PDMS stamp using a PMMA mold according to one embodiment of the present invention;

FIG. 12 is a schematic representation of the formation of an alginate mold from a PDMS mold according to one embodiment of the present invention;

FIG. 13 is a schematic representation of the formation of a collagen/PEG-DA mold from the alginate mold intermediate according to one embodiment of the present invention; and

FIG. 14 is schematic representation of a peristaltic synthetic intestine comprising a 3D hydrogel scaffold and a surrounding layer replicating naturally-occurring peristaltic actions of the smooth muscles associated with the small intestine, according to one embodiment of the present invention.

### DETAILED DESCRIPTION

The present invention provides physiologically realistic, biomimetic tissue scaffolds, as well as methods of manufacture of these scaffolds. A biomimetic material is a synthetic or man-made compound or structure that mimics (e.g., replicates, reproduces, imitates, or is similar to) a biological material or structure in its structure or function. As described in detail below, the tissue scaffold can be used as a biomimetic material to effectively and affordably imitate, in structure and/or function, a wide variety of biological materials and structures.

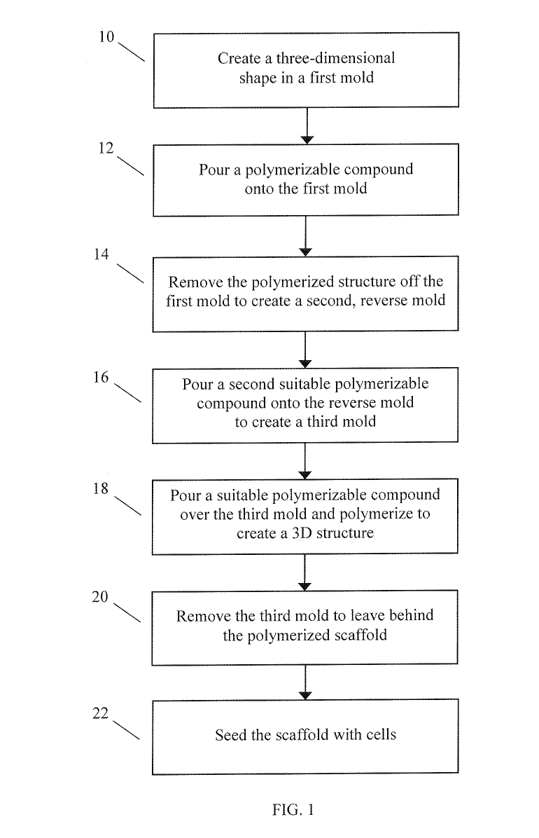

Referring now to the drawings, wherein like reference numerals refer to like parts throughout, there is seen in FIG. 1 a representative flowchart of a method of manufacturing a biomimetic tissue scaffold according to one embodiment. At step 10, a three-dimensional shape is made in a first mold. In one embodiment, the shape is made using a laser, but can be formed using any tool or equipment capable of forming a shape in a mold by removing material from the mold. The first mold is preferably plastic, but can be also be any substance, compound, or material that is capable of accepting a three-dimensional (“3D”) shape while being sufficiently rigid to maintain the shape in downstream steps of the method, and being sufficiently smooth to prevent undesirable shapes from forming.

The 3D shape formed in the first mold is any shape, size, configuration, pattern, depth, width, or other geometry capable of being formed in the mold, and further capable of being reproduced by downstream steps (i.e., capable of being adopted by a polymerizable compound). In one embodiment, the 3D shape formed in the first mold is similar to or representative of a three-dimensional geometry found in a biological system.

For example, the 3D shape in the mold can be, but is not limited to, an array of high-aspect ratio indentations. In one embodiment, a “high-aspect ratio” indicates that the height of each indentation is greater than the width of the indentation, although other configurations are possible. In one embodiment of the invention, the distance from the base of the artificial villi to the rounded tip of the villi will be greater than the width of the villi at its base, and the aspect ratio is greater than that of artificial villi created using previous methods.

At step 12 of the method, a polymerizable compound is poured onto the first mold in order to create a second mold. The monomer or compound must be capable of filling and adopting the 3D shape formed in the first mold. The compound is then made to polymerize through the use of temperature, time, a polymerizing agent, and/or any other polymerization trigger. The polymerization requirements will depend upon the particular polymerizable monomer or other polymerizing compound chosen for the second mold.

In the next step of the method, step 14, the polymerized structure is removed from the first mold, thereby creating a second, reverse mold which is used in further steps of the method. In step 16, a second polymerizable monomer or other polymerizing compound is poured onto the reverse mold in order to create a third mold. The monomer or compound is then caused to polymerize through the use of temperature, time, a polymerizing agent, and/or any other polymerization trigger. The polymerization requirements will depend upon the particular polymerizable monomer or other polymerizing compound chosen for the second mold. Once polymerized, the third mold is removed from the reverse mold and is used in downstream steps of the method. Accordingly, the second polymerizable compound used to form the third mold in step 16 is preferably any compound that can be removed in a

downstream step of the method. Examples of suitable compounds include, but are not limited to, alginate, gelatin, chitosan, collagen, poly-N-isopropylacrylamide (“poly-NIPAM”), cationic poly(ester amide) (“PEA”)-based hydrogels, polysaccharide-based polymers, poly(ethylene glycol) (“PEG”), poly(ethylene glycol) diacrylate (“PEG-DA”), polycarbonate, acrylate, and combinations thereof.

At step 18, a pre-gel solution of a polymerizable material is poured into the third mold and allowed to polymerize. Once the material has polymerized, the third mold is removed. For example, at optional step 20, the third mold is gently dissolved. In this embodiment, the third mold is used as a sacrificial layer for making the final structure. Gently dissolving the third mold eliminates the need for applying force or stress during removal of the mold, and provides a physiologically mild environment for subsequent cell culture. Examples of suitable compounds for the scaffold include, but are certainly not limited to, hydrogels, gelatin, chitosan, collagen, poly-N-isopropylacrylamide (“poly-NIPAM”), polysaccharide-based polymers, PEG, poly(ethylene glycol) diacrylate (“PEG-DA”), basement membrane proteins such as fibronectin, laminin, and entactin, and combinations thereof

Finally, at step 22, the three-dimensional structure is seeded with cells and cultured for a period of time, preferably until the entire structure is coated with living cells. Alternatively, cells are encapsulated in the polymerizable material prior to polymerization.

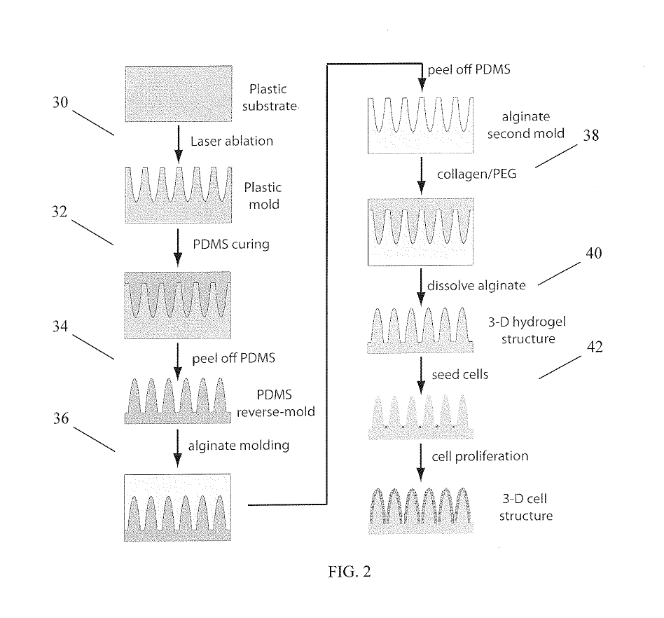

According to one embodiment of the method, as shown in FIG. 2, an array of high-aspect ratio indentations, approximately 500 micrometers deep, are made on a plastic mold using laser ablation at step 30. At step 32, polydimethylsiloxane (“PDMS”) is poured onto the plastic mold and cured to create 3D structure. A ubiquitous silicon-based organic polymer, PDMS forms a suitable three-dimensional structure after it is allowed to polymerize. As discussed above, however, the material used to create the reverse mold can be any suitable polymerizing compound. At step 34, the polymerized PDMS structure is peeled off of the plastic mold, resulting in a PDMS reverse mold. Then, at step 36, a second polymerizable solution, 2.5% calcium alginate, is poured onto the PDMS reverse mold to create the third mold. Alginate, also known as alginic acid, is a polysaccharide most commonly derived from seaweed such as brown algae. One of the many uses of alginate is as a polymerizing polymer, since it functions as an anionic polymer that binds divalent cations (such as Ca²⁺) to form a polymer network. The rate of polymerization can be controlled by varying the concentration of alginate and/or the cation used in the polymerization reaction. Once the alginate mold forms, it is removed from the PDMS mold.

Next, a pre-gel solution of the final hydrogel, collagen/PEG-DA, is poured into the alginate mold and allowed to polymerize at step 38. Collagen is the most abundant protein in the mammals, and is frequently used as a scaffold for cultures of various cell types. PEG is a synthetic, biocompatible hydrogel widely used for cell culture. Once the collagen/PEG-DA hydrogel has polymerized, the alginate mold is dissolved at step 40 using, for example, an EDTA solution. Finally, at step 42, the three-dimensional hydrogel structure is seeded with cells and cultured for a period of time, preferably until the entire hydrogel structure is coated with living cells. Alternatively, cells are encapsulated in the hydrogel prior to polymerization.

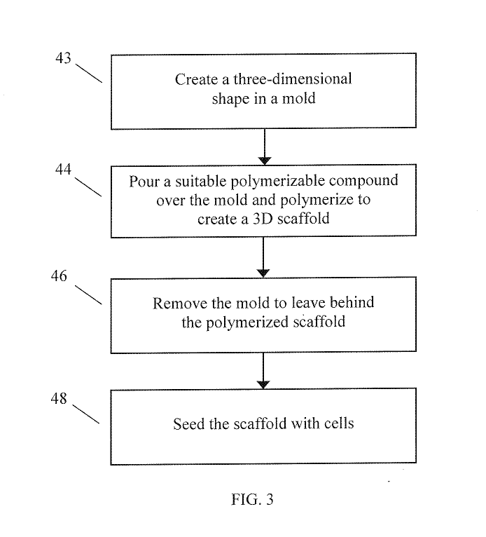

According to yet another embodiment of the method, as shown in FIG. 3, at step 43 a three-dimensional shape of any size, configuration, pattern, depth, width, or other geometry is made in a mold according to methods and techniques known in the art and described herein. At step 44, a suitable polymerizable compound is poured onto the first mold in order to create a second mold. The monomer or compound must be capable of filling and adopting the 3D shape formed in the mold. The compound is then made to polymerize through the use of temperature, time, a polymerizing agent, and/or any other polymerization trigger. The polymerization requirements will depend upon the particular polymerizable monomer or other polymerizing compound chosen for the scaffold.

Examples of suitable compounds for the 3D scaffold include, but are not limited to, polydimethylsiloxane and other silicones, hydrogels, alginate, gelatin, chitosan, collagen, poly-N-isopropylacrylamide (“poly-NIPAM”), polysaccharide-based polymers, poly(ethylene glycol) (“PEG”), poly(ethylene glycol) diacrylate (“PEG-DA”), polycarbonate, acrylate, basement membrane proteins such as fibronectin, laminin, and entactin, and combinations thereof.

In the next step of the method, step 46, the mold is removed from the 3D scaffold (or, alternatively, the 3D scaffold is removed from the mold). Finally, at step 48, the three-dimensional structure is seeded with cells and cultured for a period of time, preferably until the entire structure is coated with living cells. Alternatively, cells are encapsulated in the polymerizable material prior to polymerization.

Results

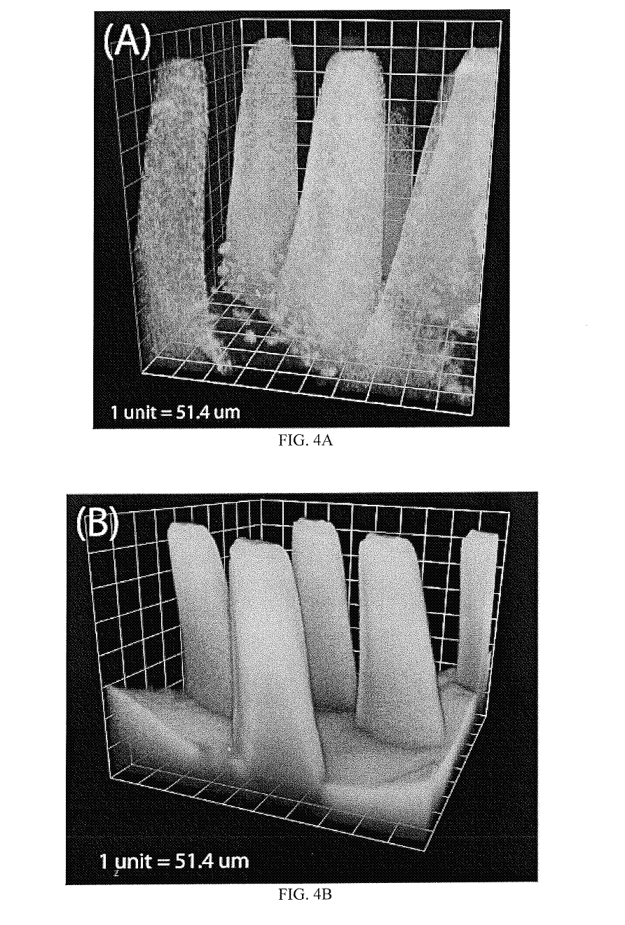

Using the method described in detail above, a scaffold mimicking the actual geometry and density of the villi structures in the human GI tract was fabricated. For example, two types of hydrogels were tested: collagen and polyethylene glycol (“PEG”). FIG. 4A shows an image of a three-dimensional villi structure made according to one embodiment of the present invention with 0.5% (w/v) collagen. FIG. 4B shows the same structure made with 20% PEG. In both cases, the height of the structure was approximately 450˜500 μm, verifying that the serial molding process described herein accurately replicates the 3D geometry of villi structures in the human GI tract.

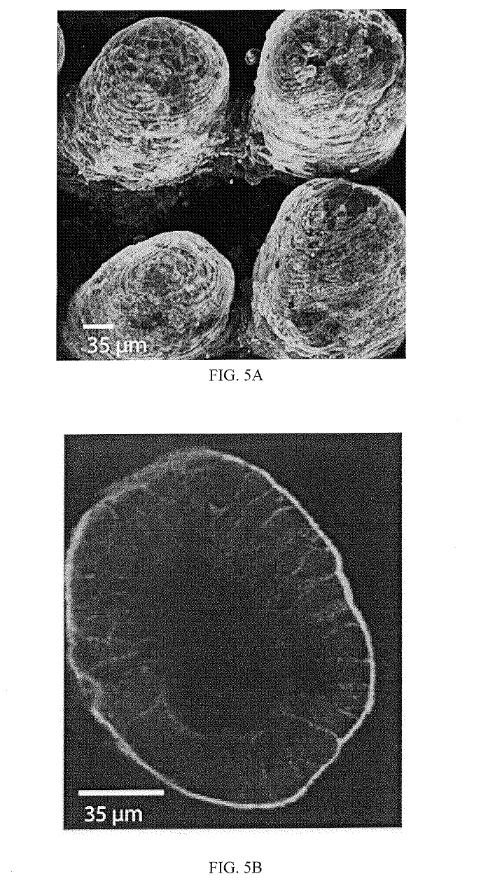

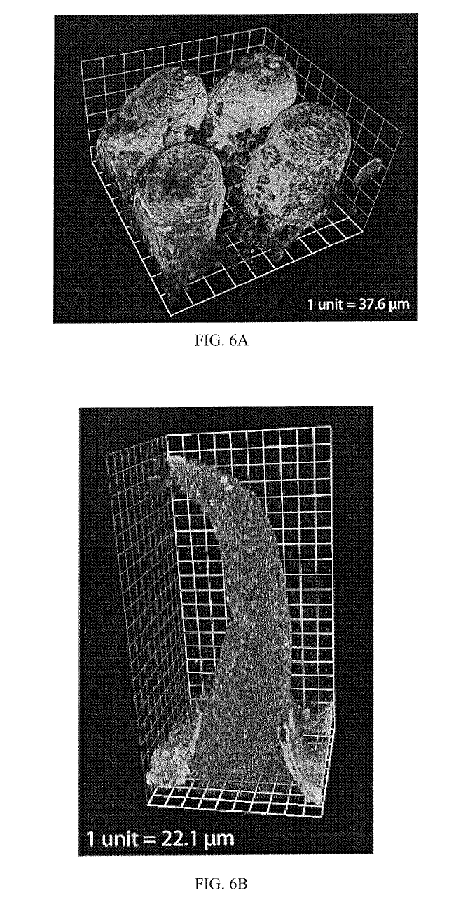

To demonstrate the viability of using the generated villi structure as a 3D scaffold for cell culture, the Caco-2 cell line—which originated from human colon adenocarcinoma and is widely used as an in vitro model of gastrointestinal epithelial cell lining in drug absorption studies—was used. Caco-2 cells were seeded onto the scaffold and cultured for up to three weeks. As the cells proliferated, they invaded and covered the collagen villi, as depicted in FIG. 5A. For visualization, the cells were fixed with formaldehyde and stained for actin and nucleic acid using Alexa Fluor® 488 phalloidin and TO-PRO-3, respectively. The overall morphology of the cell-covered collagen structure shows a striking similarity to scanning electron microscope images of human jejunal villi (not shown). An x-y slice image of stained cells, shown in FIG. 5B, revealed that the cells proliferated around the collagen scaffold, forming a uniform coverage. In this particular embodiment, after cells completely covered the collagen surface, it was observed that the height of the collagen structure was reduced to about half of the original height (approximately 250 μm), as depicted in FIG. 6A. This was caused by several factors, including the tension from the cells attached to the collagen matrix, degradation of collagen during invasion of cells into the matrix, and formation of a cell multilayer at the bottom surface. It was not, however, due to any instability of the collagen scaffold, as the scaffold remained intact while immersed in cell culture medium for three weeks without cells, as shown in FIG. 6B.

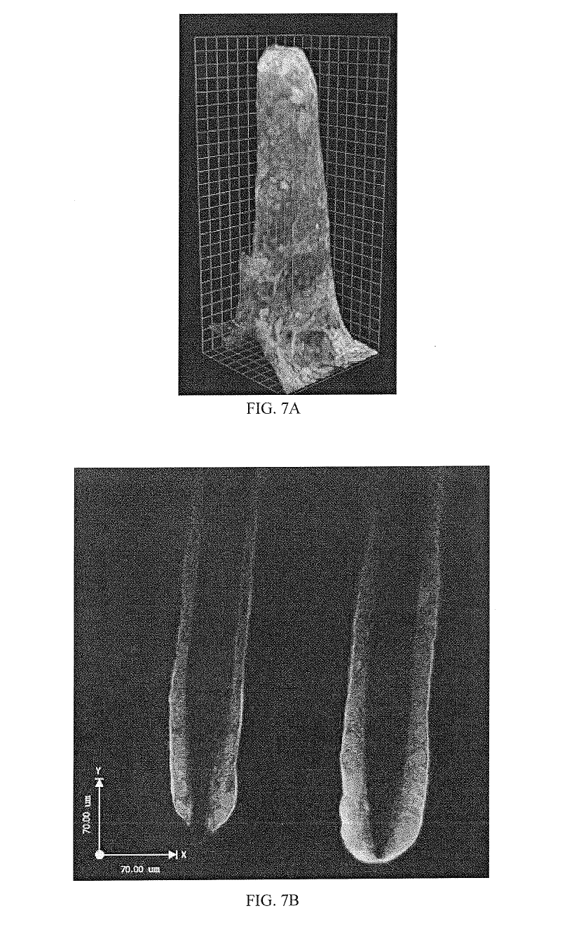

To demonstrate the viability of the embodiment described above and shown in FIG. 3, a scaffold mimicking the actual geometry and density of the villi structures in the human GI tract was fabricated from PDMS from a first mold. FIGS. 7A and 7B show an image of a three-dimensional villi structure made from PDMS. The villi structures were seeded with Caco-2 cells and cultured for up to three weeks. As the cells proliferated, they covered the PDMS villi, as depicted in FIGS. 7A and 7B.

### EXAMPLE 1

Creating the PMMA Mold and the PDMS Mold

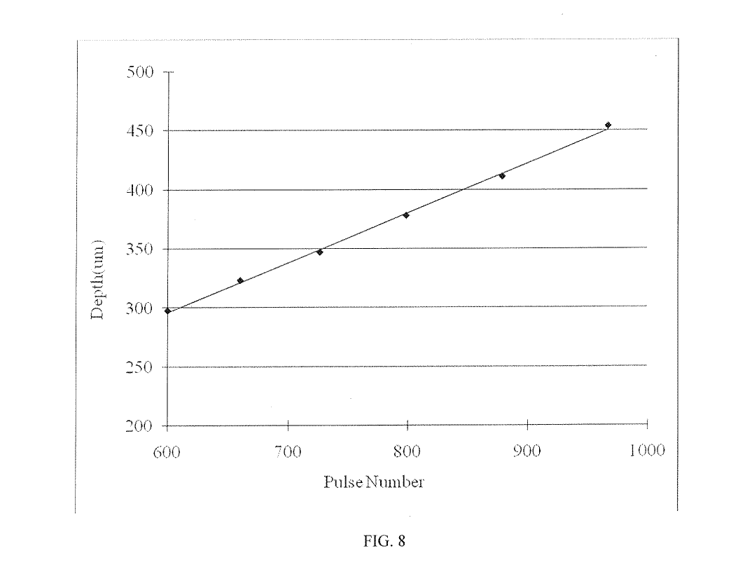

Described below are methods for creating the PMMA and PDMS molds according to one embodiment of the present invention, although it is not an exhaustive description of the possible methods of manufacture. Poly (methyl methacrylate) (“PMMA”) was purchased from Ithaca Plastics, Inc (Ithaca, N.Y.). UV laser micromachining system Resonetics Maestro 1000 (Resonetics, Nashua, N.H.) was used to fabricate high-aspect ratio indentations in PMMA. The laser energy was stabilized at 50 mJ by using energy stable function. A stainless sheet with 4 mm diameter circle was used as laser shutter. For this fabrication, a pulsed laser (at 193 nm for this experiment, although many other wavelengths are possible) was used to create indentations comparable to the depth:width ratio of villous structures. The laser pulse rate was set at 75 PPS (pulse per second), although other pulse rates are possible, and the pulse number was set to 1100, although other pulse numbers could be used. At these parameters, the average depth of the indentations was estimated to be approximately 506 μm, which was later confirmed by confocal microscopy. The distance between rows and columns was set to be 200 μm for a density of 25 indentations/mm². To measure the depth of the indentations, a drilled PMMA sheet was coated with gold by a gold sputtering system (Polaron) for 30 min to generate detectable signals. The depth was measured by Wyko® HD-3300 noncontact surface height measurement system (Veeco® Instruments Inc, Tucson, Ariz.). A linear relation was found between laser pulse number and the indentation's depth, as shown in FIG. 8. PDMS monomer and curing agent (Sylgard® 184, Dow Corning®, Midland, Mich.) were mixed at 7:1 ratio, and poured onto the PMMA with indentations. After degassing to remove bubbles and ensure PDMS prepolymer solution has filled up the indentations, the PDMS was cured at room temperature overnight. After curing, PDMS mold was slowly peeled off.

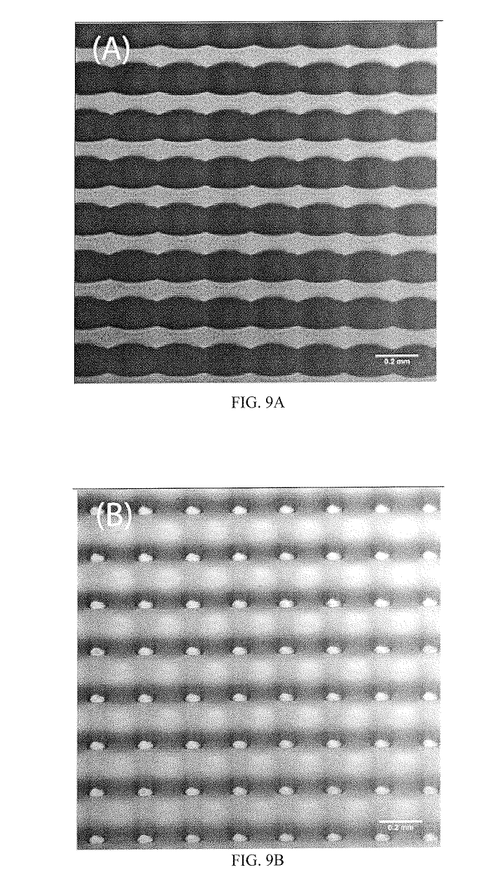

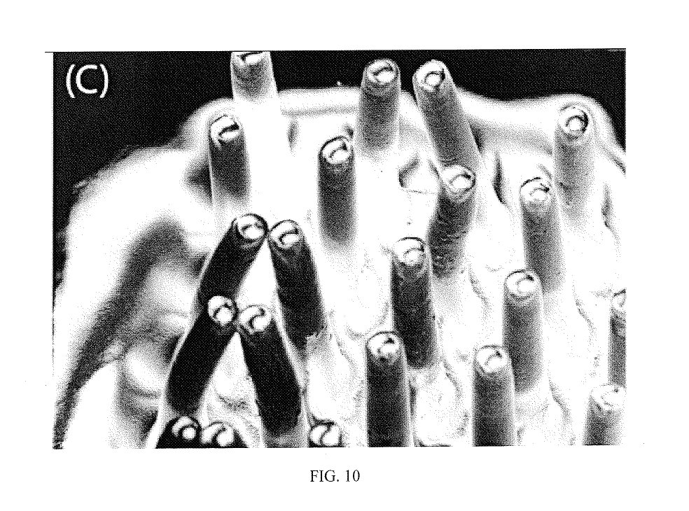

FIG. 9A, for example, depicts a PMMA mold after laser pulses created indentations at a density of approximately 25/mm², where each indentations has an oval shape due to the melting effect of the laser. The longer axis of the oval is about 200 μm, and the shorter axis is about 160 μm. FIG. 9B depicts the reverse surface of the same PMMA mold with the camera focused on the bottom of the indentations. It can be seen that the indentation size decrease as the depth increases. Lastly, FIG. 10 is a scanning electron microscope image of a PDMS structure made from the PMMA mold, after the PDMS mold is slowly peeled off the PMMA mold.

### EXAMPLE 2

Creating the Alginate Mold and the Collagen/PEG-DA Scaffold

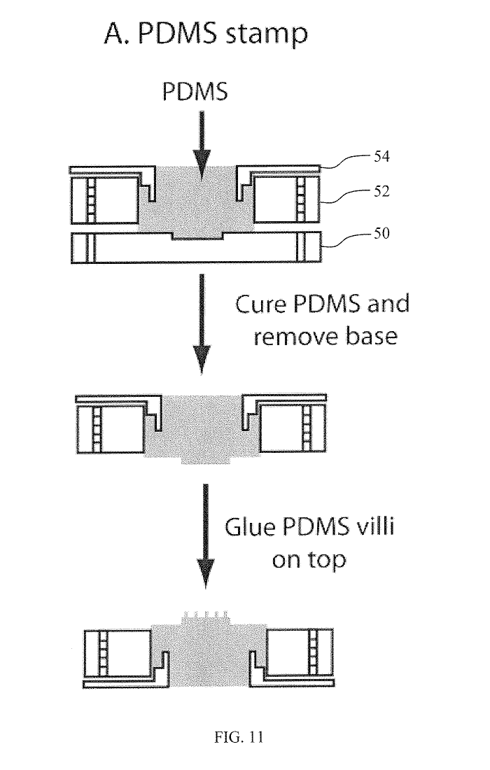

Described in detail below are methods for creating the alginate mold and the collagen/PEG-DA scaffold according to one embodiment of the present invention, although it is not an exhaustive description of the possible methods of manufacture. For fabrication of an alginate mold, for example, a PDMS stamp with villi structure is made first. An aluminum gasket was designed based on a previously reported method using a gasket for fabricating microfluidic channels in calcium alginate. It consists of a base frame, numeral 50 in FIG. 11, with a recess (7 mm×7 mm, 0.7 mm depth), a middle frame for holding PDMS (numeral 52), and the top frame (numeral 54). The three frames were secured with screws. The PDMS stamp was cured overnight at room temperature to avoid deformation of aluminum from heating. After curing, base frame 50 was removed, and the PDMS villi structure (made from the PMMA mold) was glued on top of the cured PDMS. Uncured PDMS prepolymer solution was used as glue. The whole set was left at room temperature overnight until the PDMS glue set.

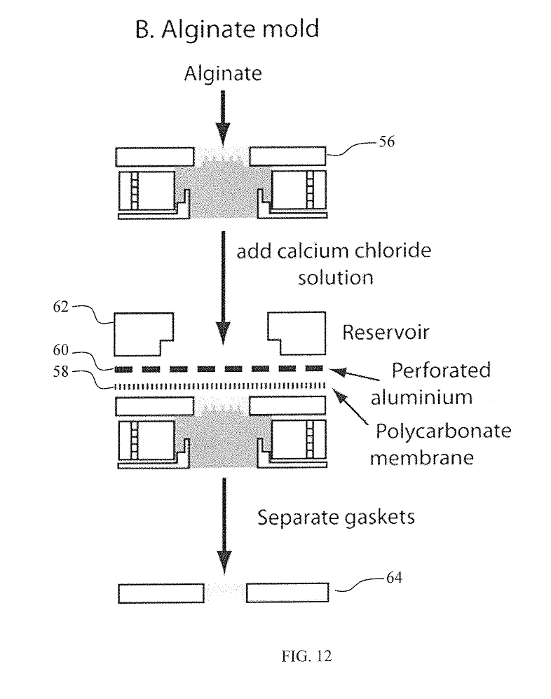

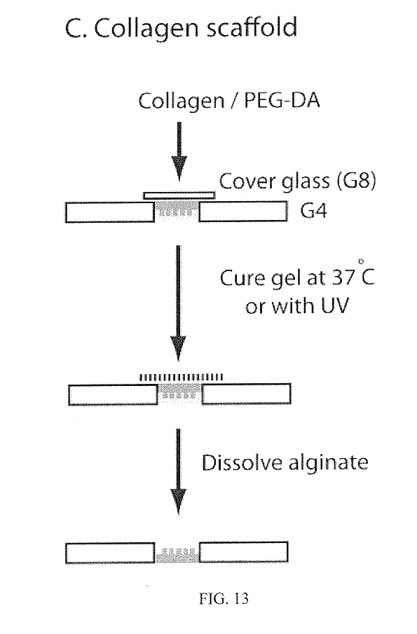

After the PDMS villi piece was fully glued, an aluminum gasket, labeled numeral 56 in FIG. 12, was secured on top of the PDMS stamp. Gasket 56, a square piece with 10 mm by 10 mm hole, is used as a gasket for holding the alginate mold. Sterile-filtered 2.5% sodium alginate (10/60 sodium alginate, FMC Biopolymer, Philadelphia, Pa.) was inserted into a hole in gasket 56. The top was covered with a polycarbonate membrane (numeral 58, preferably 8 μm pore size and 25 mm diameter, Fisher Scientific®, Pittsburg, Pa.) and a perforated aluminum piece (numeral 60) with 1 mm diameter holes. An aluminum gasket 62, which works as a reservoir for calcium chloride solution is secured on top, and 3 ml of 60 mM calcium chloride solution was inserted into the reservoir. After incubating at room temperature for 4 hours, the gasket 56 with the alginate mold, shown at 64 in FIG. 12, was separated from the other gasket pieces. Collagen or PEG-DA pre-gel solution (5 mg/ml final concentration in 0.1% acetic acid for collagen and 20% (w/v) for PEG-DA with 0.5% 2,2+-Azobis(2-methylpropionamidine)dihydrochloride as a photoinitiator) was placed in the alginate mold. Collagen pre-gel solution was neutralized with 1M NaOH and kept in ice before the insertion. Collagen was gelled by raising the temperature to 37° C., and PEG-DA was polymerized by exposure to UV for 30 minutes in a UV crosslinker (Spectronics® Corporation, Westbury, N.Y.), as shown in FIG. 13. The collagen was further crosslinked with 0.1% glutaraldehyde for 4 hours. After the gel was made, the alginate mold was dissolved using 60 mM EDTA solution for 3 hours at room temperature.

### EXAMPLE 3

Cell Seeding and Staining

After fabrication, a collagen scaffold was incubated in 5% L-glutamic acid for 48 hours at room temperature to remove the glutaraldehyde and restore the biocompatibility. Then the scaffold was washed in PBS three times, and incubated in PBS until cell seeding. Caco-2 cells were maintained in Dulbecco's Modified Eagle's Media (DMEM, Cellgro, Manassas, Va.), with 10% FBS (Invitrogen, Carlsbad, Calif.) and 1× anti-biotic anti-mycotic (Invitrogen). After trypsinization, live cell number was counted and cells were resuspended in the medium to the final concentration of 1×10⁵-5×10⁵ cells/ml. A drop of cell suspension was placed on top of the collagen scaffold and incubated for 30 minutes before medium was added. After cell seeding, the collagen scaffold was maintained in a cell culture incubator with the medium changed every two days. Depending on the initial seeding density, cells will cover the collagen scaffold in 7-10 days. After the collagen scaffold is covered, cells were fixed with formaldehyde, washed with PBS, and then stained with Alexa Fluor 488 phalloidin (Invitrogen) and TO-PRO-3 (Invitrogen). Fluorescently labeled phalloidin is a high-affinity probe for F-actin and TO-PRO-3 is a nucleic acid stain. Confocal images were taken with Leica SP2 confocal microscope (Leica Microsystems, Bannockburn, Ill.) and 3D image was rendered using Volocity (Perkinelmer, Waltham, Mass.).

Applications

The results described herein demonstrate the feasibility of the described method for creating a hydrogel scaffold mimicking the microscale geometries of biological tissues. Using alginate as a sacrificial layer is particularly advantageous since the alginate dissolving process is mild, and therefore compatible with applications involving cells. Physiologically realistic, three-dimensional models of intestinal villi may greatly improve, for example, in vitro drug absorption studies, allowing for improved predictability when compared to conventional Caco-2 monolayers. Moreover, the method will be applicable to various types of synthetic and natural hydrogels, as well as complex shapes of various biological tissues. The method and the novel hydrogel scaffold will also have significant contribution to several research disciplines, such as tissue engineering, pharmaceutical sciences, and cell biology.

Commensal bacteria living in the human gastrointestinal (“GI”) tract are indispensable for maintaining normal metabolic function. It is estimated that there are over 300 types of microorganisms living in the intestine, and these organisms have been shown to communicate with the human epithelial cells that line the GI tract. This communication consists of hormones and small molecules that pass from the epithelia to the bacteria and metabolic products that pass from the bacteria to the epithelia. As with any system of communication there are rules governing information transfer between the commensal bacteria and their host. These rules are only recently being understood, but there is mounting evidence that the level of access commensal bacteria have to epithelia is greater than previously believed. In addition to aiding with digestion, gut bacteria play a vital role in the development of infant GI tracts. With such an integrated role in human physiology, commensal bacteria are ideally situated to sense changes in the environment of the gut.

One embodiment of the present invention is an intestinal tubular reactor, or a “gut-tube reactor” system, which will be useful in studying this commensal interaction between bacteria and the human GI tract. According to one embodiment, the gut-tube reactor system is composed of fabricated villi ‘rolled’ to form a hollow spherical tube. For example, the “3D cell structure” depicted as the final step in FIG. 2 can be a sheet that is rolled to form a hollow spherical tube similar to a small intestine.

The gut-tube reactor system enables rapid, high throughput testing and characterization of gut interactions in a potentially more “human-like” system without the need for expensive and slow mouse models, and will therefore allow for characterization and optimization to address a variety of diseases that include diabetes, multiple sclerosis, irritable bowel syndrome, cholera, and cancer as well as allow the study of intestinal nutrient, drug, and metabolite transport as well as study of beneficial and non-beneficial intestinal commensal bacteria in a more natural environment, among many other uses.

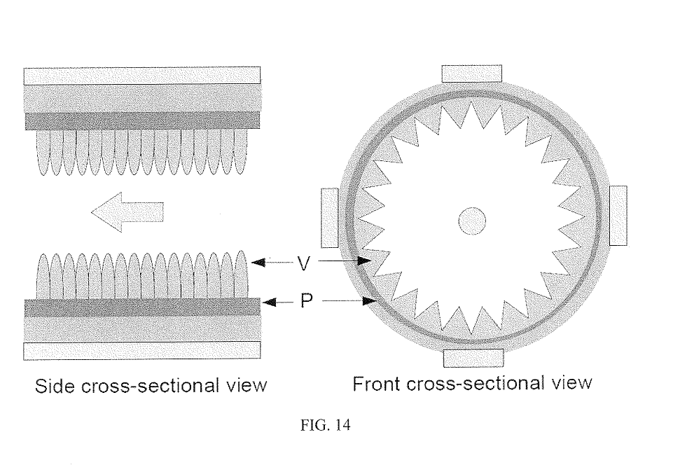

The gut-tube reactor can also facilitate long-term studies of interactions between gut micro- and macro-organisms (such as parasitic worms) and the epithelia; something not currently possible with simpler co-culture models. The gut-tube reactor can consist of various polymer scaffolds modified to house human gut cells (e.g. epithelia). These “cell scaffolds” can be arranged into the gut-tube reactor so as to mimic the structure of the GI tract on a micro scale. The architecture of the gut-tube reactor can closely resemble that of the upper GI tract in that it will be a three dimensional tube of cells, as shown in FIG. 14 (where “V” indicates cells and “P” indicates the underlying matrix). Commensal bacteria can be added into the tube to study their interactions with the epithelia that will be embedded in the tube walls or use these tubes to study nutritional uptake or diffusion. Each gut tube can be fed semi-continuously, in the same manner that the actual GI tract is fed by intermittently consumed meals. The gut tube can be used to test the responses of the epithelia to the commensal bacteria under various conditions over time. Some polymer scaffolds can mimic the peristaltic movements of the GI tract. To our knowledge, no other group is working developing novel reactor systems to study intestinal ecology.

The 3D cell gut tube model is an improvement over current systems. Some of the limitations of 2D cultures for studying bacterial/epithelial interactions include: the lack of a protective layer for the epithelia similar to the intestinal mucosa; the absence of intestinal degradative enzymes (such as DPP-IV) and the inability to maintain epithelia in the presence of much more rapidly-growing bacteria. The scaffolds maintain 3D growth of mammalian cell growth. Further, these scaffolds could be functionalized with enzymes such as DPP-IV that could serve to better represent gut conditions. Finally, these scaffolds allow for bidirectional feeding of a co-culture such that the epithelia are maintained basolaterally and the commensal bacteria are maintained from the surface.

One embodiment of the 3D gut-tube reactor is a peristaltic synthetic intestine in which the 3D hydrogel scaffold is used in conjunction with a mechanism to replicate naturally-occurring peristaltic actions of the smooth muscles associated with the small intestine. For example, the seeded hydrogel scaffold can be surrounded by a cuff or other malleable structure that mechanically replicates peristalsis. A computer can be used to activate controllers programmed to follow intestinal peristaltic algorithms. Perfusion of the device both basolaterally and apically will allow for both nutrient supplementation and sample gathering on both sides of the epithelial cells. This will provide data on both the interactions between bacteria and epithelia as well as the epithelial response to rest of the body.

The peristaltic synthetic hydrogel scaffold will be utilized to test various flow fields and media conditions over different time scales to study the effects on bacterial diversity, bacterial communication and epithelial response. In addition to culturing the four types of enterocyte cells (Paneth, Absorptive, Enteroendocrine and Goblet), the peristaltic synthetic hydrogel scaffold will also house bacterial cultures of various compositions.

Unless otherwise defined herein, scientific and technical terminologies employed in the present disclosure shall have the meanings that are commonly understood and used by one of ordinary skill in the art. Further, unless otherwise required by context, it will be understood that singular terms shall include plural forms of the same and plural terms shall include the singular. In particular, the singular forms “a” and “an” include the plural unless the context clearly indicates otherwise.

Unless otherwise expressly specified, all of the numerical ranges, amounts, values and percentages such as those for quantities of materials, durations of times, temperatures, operating conditions, ratios of amounts, and the like shall be understood as modified in all instances by the term “about.” As a result, unless there is indication to the contrary, the numerical parameters set forth in the present disclosure and attached claims are approximations that can vary as desired.

Although the present invention has been described in connection with one embodiment, it should be understood that modifications, alterations, and additions can be made to the invention without departing from the scope of the invention as defined by the claims.

What is claimed is:

1. A method for making a three-dimensional biomimetic scaffold capable of supporting growth of a cell, the method comprising the steps of:
forming a first three-dimensional shape in a first mold;
filling at least a portion of the three-dimensional shape in the first mold with a first polymerizable compound;
causing said first polymerizable compound to polymerize to form a three-dimensional scaffold, wherein said three-dimensional scaffold is complementary to said three-dimensional shape; and
removing said three-dimensional scaffold from said first mold.

2. The method of claim 1, wherein said first mold comprises a plastic.

3. The method of claim 1, wherein said three-dimensional shape is formed using laser ablation.

4. The method of claim 1, wherein said first mold comprises a plurality of three-dimensional indentations.

5. The method of claim 4, wherein each of said plurality of indentations has a maximum height and a maximum width, and further wherein for a majority of said plurality of indentations the maximum height of said indentation is greater than the maximum width of said indentation.

6. The method of claim 5, wherein a majority of said plurality of indentations have a conical shape.

7. The method of claim 1, wherein said first polymerizable compound comprises a silicone.

8. The method of claim 7, wherein said first polymerizable compound comprises polydimethylsiloxane.

9. The method of claim 1, further comprising the step of seeding said first polymerizable compound with a cell at some point prior to the step of causing said first polymerizable compound to polymerize to form a three-dimensional scaffold.

11. A method for making a three-dimensional biomimetic scaffold capable of supporting growth of a cell, the method comprising the steps of:
filling at least a portion of a three-dimensional shape formed in a first mold with a first polymerizable compound;
causing said first polymerizable compound to polymerize to form a second mold, wherein at least a portion of said second mold comprises a first structure, said first structure being complementary to said three-dimensional shape;
removing said second mold from said first mold;
using said second mold to form a third mold from a second polymerizable compound;
removing said third mold from said second mold; and
using said third mold to form a three-dimensional scaffold from a third polymerizable compound, wherein said three-dimensional scaffold is complementary to said three-dimensional shape.

12. The method of claim 11, further comprising the step of:
removing the third mold away from the three-dimensional scaffold.

13. The method of claim 11, wherein said first mold comprises a plastic.

14. The method of claim 11, wherein said first mold comprises poly (methyl methacrylate).

15. The method of claim 11, further comprising the step of:
forming the first three-dimensional shape in the first mold.

16. The method of claim 15, wherein said three-dimensional shape is formed using laser ablation.

17. The method of claim 11, wherein said first mold comprises a plurality of three-dimensional indentations.

18. The method of claim 17, wherein each of said plurality of indentations has a maximum height and a maximum width, and further wherein for a majority of said plurality of indentations the maximum height of said indentation is greater than the maximum width of said indentation.

19. The method of claim 18, wherein a majority of said plurality of indentations have a conical shape.

20. The method of claim 11, wherein said first polymerizable compound comprises a silicone.

21. The method of claim 20, wherein said first polymerizable compound comprises polydimethylsiloxane.

22. The method of claim 11, wherein said second polymerizable compound comprises alginate.

23. The method of claim 11, wherein the step of removing the third mold away from the three-dimensional scaffold comprises addition of a chelator.

24. The method of claim 23, wherein said chelator is ethylenediaminetetraacetic acid.

25. The method of claim 11, wherein said second polymerizable compound is selected from the group consisting of a hydrogel, alginate, gelatin, chitosan, collagen, poly-N-isopropylacrylamide, a polysaccharide-based polymer, poly(ethylene glycol), poly(ethylene glycol)diacrylate, and combinations thereof.

26. The method of claim 11, wherein said third polymerizable compound comprises a hydrogel.

27. The method of claim 26, wherein said hydrogel is selected from the group consisting of gelatin, chitosan, collagen, poly-N-isopropylacrylamide, a polysaccharide-based polymer, poly(ethylene glycol), poly(ethylene glycol)diacrylate, laminin, fibronectin, entactin, and combinations thereof.

28. The method of claim 11, wherein said third polymerizable compound further comprises a basement membrane protein.

29. The method of claim 11, further comprising the step of seeding said third polymerizable compound with a cell at some point prior to the step of using said third mold to form said three-dimensional hydrogel scaffold.

30. The method of claim 11, further comprising the steps of seeding the three-dimensional scaffold with a cell; and
incubating the cell.

31. The method of claim 11, further comprising the step of:
using said three-dimensional scaffold for pharmacological testing.

32. The method of claim 11, further comprising the step of:
using said three-dimensional scaffold to examine a biological process.

33. The method of claim 11, further comprising the step of:
using said three-dimensional scaffold for toxicological testing.

34. A system for making a three-dimensional biomimetic scaffold capable of supporting growth of a cell, the system comprising:
a first mold comprising a three-dimensional shape;
a second mold formed from said first mold using a first polymerizable compound; and
a third mold formed from said second mold using a second polymerizable compound, wherein said third mold is configured to form a three-dimensional scaffold complementary to said three-dimensional shape.

35. The system of claim 34, wherein the polymerization of said second polymerizable compound is reversible.

36. The system of claim 34, wherein said first mold comprises a plurality of three-dimensional indentations.

37. The system of claim 36, wherein each of said plurality of indentations has a maximum height and a maximum width, and further wherein for a majority of said plurality of indentations the maximum height of said indentation is greater than the maximum width of said indentation.

38. The system of claim 34, wherein said second polymerizable compound is selected from the group consisting of a hydrogel, alginate, gelatin, chitosan, collagen, poly-N-isopropylacrylamide, a polysaccharide-based polymer, poly(ethylene glycol), poly(ethylene glycol)diacrylate, and combinations thereof.

39. The system of claim 34, wherein said third polymerizable compound comprises a hydrogel.

40. The system of claim 39, wherein said hydrogel is selected from the group consisting of gelatin, chitosan, collagen, poly-N-isopropylacrylamide, a polysaccharide-based polymer, poly(ethylene glycol), poly(ethylene glycol)diacrylate, laminin, fibronectin, entactin, and combinations thereof.

41. The system of claim 34, wherein said third polymerizable compound further comprises a basement membrane protein.

42. The system of claim 34, further comprising:
a cell seeded on or in said three-dimensional scaffold.

43. A three-dimensional scaffold formed by the method of claim 1.

44. The three-dimensional scaffold of claim 43, wherein said scaffold comprises a polymerized hydrogel.

45. The three-dimensional scaffold of claim 43, further comprising:
a cell seeded on or in said scaffold.

46. The three-dimensional scaffold of claim 43, wherein said scaffold comprises a plurality of three-dimensional shapes.

47. The three-dimensional scaffold of claim 46, wherein each of said plurality of three-dimensional shapes comprises a high-aspect ratio of height to width.

48. A method for making an intestinal reactor, the method comprising the steps of:
forming a biomimetic scaffold comprising a plurality of villi;
seeding at least one of said villi with a cell; and
forming a hollow tube from said seeded biomimetic scaffold, said hollow tube having an interior surface and an exterior surface.

49. The method of claim 48, wherein said villi are located on the interior surface of said hollow tube.

50. The method of claim 48, wherein said villi are located on the exterior surface of said hollow tube.

51. The method of claim 48, further comprising the step of:
adding a microorganism to said intestinal reactor.

52. The method of claim 48, further comprising the step of:
adding nutrients to said intestinal reactor.

53. The method of claim 48, further comprising the step of:
using said intestinal reactor for pharmacological testing.

54. The method of claim 48, further comprising the step of:
using said intestinal reactor to examine an intestinal process.

55. An intestinal reactor formed by the method of claim 48.
